(19) United States
(12) Patent Application Publication
(10) Pub. No.: US 2011/0311635 A1
(43) Pub. Date: Dec. 22, 2011

(54) HOLLOW METAL OXIDE SPHERES AND NANOPARTICLES ENCAPSULATED THEREIN

(75) Inventors: Galen D. Stucky, Santa Barbara, CA (US); Nicholas C. Strandwitz, Santa Monica, CA (US)

(73) Assignee: THE REGENTS OF THE UNIVERSITY OF CALIFORNIA, Oakland, CA (US)

(21) Appl. No.: 13/148,763

(22) PCT Filed: Feb. 12, 2010
(86) PCT No.: PCT/US2010/024082

§ 371 Date: Aug. 10, 2011

Related U.S. Application Data
(60) Provisional application No. 61/151,969, filed on Feb. 12, 2009.

(51) Int. Cl. A61K 9/14; C01B 13/00; B01J 35/08; B01J 23/52; B01J 23/42; B01J 23/46; B01J 21/06; B01J 21/08; B01J 31/06; B01J 23/44; B01J 23/50; B01J 23/755; B01J 37/02; B01D 53/62; B01D 53/56; C03C 25/68; C07C 27/10; B82Y 40/00; B82Y 5/00; B82Y 30/00

(52) U.S. Cl. 424/490; 568/300; 423/592.1; 423/263; 502/300; 502/349; 502/344; 502/339; 502/325; 502/304; 502/242; 502/240; 502/159; 502/347; 502/337; 423/247; 423/239.1; 216/55; 977/811; 977/888; 977/906; 977/902

(57) ABSTRACT

A nanoparticle including a Group 3 atom-containing shell. In various embodiments, the nanoparticle includes a metal or metal catalyst-containing core, or a substantially metal-free core. In other embodiments, the nanoparticle shell is hollow. A method of preparing the nanoparticle and methods of using such particles are also provided.

### CROSS REFERENCE TO RELATED APPLICATIONS

This application claims priority under 35 U.S.C. §119 to U.S. Patent Application No. 61/151,969, filed Feb. 12, 2009, the disclosure of which is incorporated herein by reference in its entirety.

### FIELD OF THE DISCLOSURE

The invention relates to nanomaterial compositions and methods.

### BACKGROUND

The performance and therefore the applicability and lifetime of inorganic nanoparticles (“NPs”) in catalysis are limited by the compositions and geometries of materials currently available. NP catalysts typically consist of two materials: the nanoparticle catalyst and the support. The support acts to separate the NP catalysts from one another and strongly influences the catalytic properties of the overall material.

### SUMMARY

The disclosure provides a nanomaterial comprising a Group 3 atom-containing shell. In particular embodiments, the shell is a Ce atom-containing shell. In some embodiments, the nanoparticle comprises a Group 3 atom- and Group 4 atom-containing shell, with particular embodiments comprising a Ce atom- and Zr atom-containing shell. The nanoparticle of some embodiments can comprise a metal-containing core, a metal catalyst-containing core, or a substantially metal-free core. Or, the nanoparticle can comprise a hollow shell. The shell of any nanomaterial of the disclosure can be a porous shell.

The disclosure provides a nanosphere comprising a Group 3 or Group 3/Group 4 metal oxide shell. In one embodiment, the shell comprises cerium. In another embodiment, the shell comprises cerium and zirconium. In yet another embodiment, the shell comprises CeO₂. In a further embodiment, the shell comprise ZrO₂. In a specific embodiment, the shell comprise CeₓZR₁₋ₓO₂ wherein 1>x>0.5. The nanosphere may further comprising a non-metallic core encapsulated by the shell. In one embodiment, the non-metallic core comprises SiO₂, polymethacrylate or polystyrene. In a further embodiment, a metallic core is encapsulated by the shell. In one embodiment, the metal core comprises a metal selected from the group consisting of Au, Pd, Ag, Pt, Ni, Ru, or an alloy thereof. In yet another embodiment, the nanosphere is hollow. In one embodiment, the nanosphere comprises a shell of CeO₂ or CeₓZr₁₋ₓO₂ wherein 1>x>0.5. In some embodiments, the shell is porous. In embodiments wherein the shell encapsulates a metal particle the nanosphere comprises general formula M@CeₓZr₁₋ₓO₂, wherein 1>x>0.5, and wherein M comprises a noble metal and “@” refers to the encapsulation.

The disclosure also provides a method of preparing a nanomaterial of the disclosure. The method includes mixing a core nanoparticle and a reagent comprising a compound of a Group 3 atom under conditions sufficient to form a Group 3 atom-containing shell around the core nanoparticle. In some embodiments, the Group 3 atom is Ce. In certain embodiments, the method further comprises mixing a compound of a Group 4 atom with the Group 3 atom-containing shell, which results in the formation of a Group 3 atom- and Group 4 atom-containing shell around the core nanoparticle. In particular embodiments, the Group 3 atom is Ce and the Group 4 atom is Zr. The core nanoparticle of various embodiments can comprise a metal or a metal catalyst, or be substantially free of metal. The method can further include etching away all or at least part of the core nanoparticle.

The disclosure also provides a method of making a nanosphere of the disclosure comprising mixing a core nanoparticle and a reagent comprising a compound of a Group 3 atom under conditions sufficient to form a Group 3 atom-containing shell around the core nanoparticle. In one embodiment, the Group 3 atom is Ce. The method can further comprise mixing the Group 3 atom-containing shell with a compound of a Group 4 atom so as to form a Group 3 atom- and Group 4 atom-containing shell. In a further embodiment, the Group 3 atom is Ce and the Group 4 atom is Zr. In yet a further embodiment, the core can comprise a metal. In one embodiment, the core comprises Au, Pd, Ag, Pt, Ni, Ru, or an alloy thereof. In yet another embodiment, the core comprises a metal catalyst. In a further embodiment, the metal catalyst is Au, Pd, Pt, Ru, or RuO₂. In yet another embodiment, the core nanoparticle is substantially free of metal. The method may further include etching away at least part of the core nanoparticle.

In a specific embodiment, the disclosure provices a method of making a nanosphere of comprising cerium or cerium and zirconium, the method comprising: forming SiO₂ particles; suspending the SiO₂ particles in an aqueous mixture of cerium nitrate to coat the SiO₂ particles; calcining the coated SiO₂ particles to form spheres with a CeO₂ shell; and etching SiO₂ from the spheres. In one embodiment, a heterogeneous or homogenous mixture of metal nanoparticles or a metal oxide nanoparticles are included during formation of the SiO₂ particles. In a further embodiment, the metal nanoparticles comprise Au, Ag, Pd, Pt or any combination thereof. In yet another embodiment, the nanosphere has an inner diameter of less than 500 nm, the outer layer has a thickness of less than 50 nm, and the outer layer has a pore size of less than 5 nm.

The disclosure also provides a catalytic process comprising carrying out a chemical reaction in the presence of any nanomaterial described herein, where the chemical reaction is catalyzed by the nanoparticle or the shell of the nanomaterial or both. In some embodiments, the chemical reaction can involve the oxidation of a hydrocarbon, the oxidation of CO, or the reduction of a nitrogen oxide, or any combination thereof. Also provided is a catalytic device—such as used in the output stream of a waste combustion facility or a coal power plant-comprising any nanoparticle described herein. In certain embodiments, the catalytic device is a catalytic converter. Additionally, a nanoparticle is provided that includes a pharmaceutical compound encapsulated by the nanoparticle shell.

### BRIEF DESCRIPTION OF THE DRAWINGS

FIG. 1 is an illustration of a synthesis scheme for the synthesis of (A) hollow @ CeₓZr₁-xO₂ nanoparticles and (B) M@hollow CexZr₁-xO₂ nanoparticles. Here M=Au.

FIG. 2 is panel of TEM micrographs of SiO₂ templates, SiO₂@CeO₂ particles, and hollow @CeO₂ particles.

FIG. 3 is a TEM micrograph of SiO₂@CeZrO₂, here x=0.8 by EDS analysis.

FIG. 4 is a TEM micrograph of CeO₂ spheres treated at 750° C. for 5 hours.

FIG. 5 is a panel of TEM micrographs of Au@SiO₂@CeO₂ catalyst particles.

FIG. 6 is a graph showing nitrogen adsorption-desorption isotherms for @CeO₂ (open squares), SiO₂@CeO₂ particles (closed squares), and SiO₂ template spheres (closed circles).

FIG. 7 is a graph showing temperature-programmed catalytic activity of Au@CeO₂ and @CeO₂ spheres for CO oxidation.

FIG. 8 shows a generalized schematic of conventional and core-shell (encapsulated) supported nanoparticle catalysts. The same compositions of material are present (e.g. Au metal nanoparticle and cerium oxide support), but the geometry is different.

### DETAILED DESCRIPTION

As used herein and in the appended claims, the singular forms “a,” “and,” and “the” include plural referents unless the context clearly dictates otherwise. Thus, for example, reference to “a particle” includes a plurality of such particles and reference to “the sphere” includes reference to one or more spheres, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

Generally as used herein, “nanomaterials” refers to nanoparticles, nanospheres, nanowires, nanotubes and combinations, complexes, encapsulations and the like thereof. As used herein, “nanoparticle” refers to a solid particle with a diameter in the nanometers (nm). As used herein, “nanosphere” refers to a substantially hollow particle with a diameter in the nanometers. The nanosphere need not be perfectly spherical and may be oblong, substantially cuboidal and the like.

Nanoparticles (NPs) used in catalysis are strongly influenced by the type of support associated with the nanoparticle. For example, gold (Au) NPs dispersed on aluminum oxide are typically not active toward oxidation reactions. However, Au NPs on cerium oxide are quite active toward several oxidation reactions. Some reasons for this support-dependent activity include, but are not limited to, (1) electronic effects on the NP by the supporting metal oxide, (2) reactant and product adsorption and desorption rates by the supporting metal oxide.

Inorganic NP catalysts are significantly limited in applications due to the temperature- or reaction-induced sintering (or “ripening”) of NP catalyst particles. Sintering of NP catalysts increases particle size, decreases surface area, and typically decreases surface-area-normalized catalytic activity. That is, catalytic performance of the material is degraded or halted entirely during and after sintering. Thus, ripening of a NP catalyst causes a decrease in catalytic activity and is thus unfavorable. Metal NPs encapsulated in ZrO₂, TiO₂, and SiO₂ have been shown to be resistant to ripening. Because the catalyst support (including cases in which the support is also the encapsulant) dictates the activity of the metal NP catalyst.

The disclosure provides nanomaterials comprising a nanoparticle having a shell of any one or more group 3 atoms or rare earth atoms, such as scandium, yttrium, lanthanum, actinium, cerium or other lanthanide. The nanomaterial can further comprise a shell with a mixture of a group 3 atom or rare earth atoms and a group 4 atom (e.g., titanium and/or zirconium). Typically the shell will be in an oxide form.

Nanomaterials include, for example, oxides and/or nitrides of elements from columns 2-15 of the Periodic Table. Specific compounds that may be used as nanomaterials include, but not limited to, aluminum cerium oxide, aluminum nitride, aluminum oxide, aluminum titanate, antimony(III) oxide, antimony tin oxide, barium ferrite, barium strontium titanium oxide, barium titanate(FV), barium zirconate, bismuth cobalt zinc oxide, bismuth(III) oxide, calcium titanate, calcium zirconate, cerium(IV) oxide, cerium(rV) zirconium(IV) oxide, chromium(III) oxide, cobalt aluminum oxide, cobalt(II, III) oxide, copper aluminum oxide, copper iron oxide, copper(II) oxide, copper zinc iron oxide, dysprosium(III) oxide, erbium(III) oxide, europium(III) oxide, holmium(III) oxide, indium(III) oxide, indium tin oxide, iron(II, III) oxide, iron nickel oxide, iron(III) oxide, lanthanum(III) oxide, magnesium oxide, manganese(II) titanium oxide, nickel chromium oxide, nickel cobalt oxide, nickel(II) oxide, nickel zinc iron oxide, praseodymium(III, IV) oxide, samarium(III) oxide, silica, silicon nitride, strontium ferrite, strontium titanate, tantalum oxide, terbium (III, IV) oxide, tin(IV) oxide, titanium carbonitride, titanium(IV) oxide, titanium silicon oxide, tungsten (VI) oxide, ytterbium(III) oxide, ytterbium iron oxide, yttrium(III) oxide, zinc oxide, zinc titanate, and zirconium(IV) oxide. It should be understood that the above-listed materials may include minor amounts of contaminants and/or stabilizers (e.g., water and/or acetate) when obtained commercially or synthesized. Nanomaterials used for nanocomposites may be selected based on a variety of properties including, but not limited to, refractive index and hardness. Table 1 compares the bulk hardness and refractive indices of several commercially available nanomaterials.

The shell substantially surrounds a core nanoparticle or may be hollow. The core nanoparticle can be any metal including nobel metals. Nanoparticle cores useful in the disclosure can comprise, for example, a metal which exhibits a low bulk resistivity. Non-limiting examples of metals for use in the disclosure include transition metals as well as main group metals such as, e.g., silver, gold, copper, nickel, cobalt, palladium, platinum, indium, tin, zinc, titanium, chromium, tantalum, tungsten, iron, rhodium, iridium, ruthenium, osmium and lead. Non-limiting examples of commonly used metals in nanoparticles include silver, gold, copper, nickel, cobalt, rhodium, palladium and platinum.

As used herein, “nanoparticle” refers to a particle having dimensions that are less than 1,000 nanometers, and in particular having a size range of about 2 to about 500 nanometers. The nanoparticle can be any shape such as spheroid or cuboid. In some embodiments, the nanoparticle has a spheroidal shape. As used herein, the term “shell” (sometimes depicted herein as a “@”) refers to the surface layer of a nanomaterial of the disclosure. A nanomaterial comprising a shell and including a core that contains solids is referred to as a core-shell nanoparticle. A shell is called “hollow” or “empty” if it has a core that lacks solid material or lacks a core. In different embodiments, a core can be completely or partially filled with solids.

In various embodiments, a nanomaterial is provided having a shell comprising any one or more Group 3 atoms, or rare earth atoms, such as scandium, yttrium, lanthanum, actinium, or cerium or another lanthanide. In certain embodiments, the shell also comprises any Group 4 atom such as titanium or zirconium. In particular embodiments, the shell comprises an oxide of any Group 3 element and/or an oxide of any Group 4 element. In general, a shell can comprise any combination of one or more Group 3 atoms, or rare earth atoms, and/or any Group 4 atom.

In some embodiments, the core of the nanoparticle comprises a metal, usually in the form of a metal nanoparticle. Examples of metals include, but are not limited to, Au, Pd, Ag, Pt, Ni, Ru, and alloys thereof. With some embodiments, the core comprises a metal oxide such as RuO₂, CuO₂, ZrO₂, TiO₂, Al₂O₃, CeO₂, Nb₂O₅ or MnO₂. In particular embodiments, the metal or metal oxide is a metal catalyst such as Au, Pd, Pt, Ru or RuO₂.

The compositions of the disclosure can comprise homogenous nanoparticle, mixtures of two or more different metal nanoparticles (e.g., a heterogeneous mixture) and/or may comprise nanoparticles wherein two or more metals are present in a single nanoparticle, for example, in the form of an alloy or a mixture of these metals. Non-limiting examples of alloys include Ag/Au, Ag/Ni, Ag/Cu, Pt/Cu, Ru/Pt, Au/Pt and Ag/Co. Also, the nanoparticles may have a core-shell structure made of two different metals.

In other embodiments, the core of the nanoparticle is free or substantially free of metal and comprises solids other than metal. Examples of such solids include, but are not limited to, SiO₂, polymethylmethacrylate, and polystyrene. By “substantially free” is meant that metal (if any) present in the core does not affect the function or intended use of the nanoparticle.

SiO₂, methyacrylate polymers and Polystyrene beads are useful for generating hollow nanomaterials of the disclosure. Polystyrene beads are attractive nanoscale templates since they are inexpensive and their size is easily varied. Furthermore their surface can be functionalized by chemical and physical techniques. Finally they are well-suited to make hollow particles since the polystyrene template can easily be removed by calcination or dissolution. Calcination can remove the cores to generate hollow nanoparticles.

Metal and non-metal nanoparticles in the core can range in size from about 1 to about 100 nanometers, with a typical size range of about 2 to about 10 nanometers.

The disclosure also provides a method to generate a nanomaterial composition of the disclosure comprising monodisperse and hollow Group 3 or Group 3 and Group 4 atom-containing spheres, and in particular cerium-zirconium oxide (ceria-zirconia) spheres, with and without metal nanoparticles (NPs) encapsulated in the core. This method allows for a number of parameters to be tuned independently such as, for example, the core NP size and composition, the shell diameter, thickness and porosity. For example, the nanomaterial composition of the disclosure comprises a shell that may act as a catalyst independent of a core that may also act as a catalyst. In a particular embodiment, a ceria shell act as a catalyst independently or in conjunction with an encapsulated metal NP. Although ceria catalysts and metal NP catalysts supported on ceria have previously been used, hollow ceria spheres and the metal NPs encapsulated in ceria spheres represent a new geometry which is scientifically and industrially relevant. FIG. 8 contrasts conventional structures with the core-shell structures of this disclosure.

The disclosure also provides a method for the formation of hollow porous spheres comprising a Group III or Group III and Group IV oxide shell. IN one embodiment, the disclosure provides a method of making a cerium oxide and cerium-zirconium oxide shell. Further embodiments include a process for encapsulating a metal or non-metal NP catalyst (such as, for example, gold nanoparticle(s)) inside the shell (e.g., of a cerium oxide and cerium-zirconium oxide spheres). The advantages of this process and material include, for example: (1) the oxide shell (e.g., a cerium zirconium oxide shell) and encapsulated catalyst are stable against ripening at high temperature (i.e., >700° C.); (2) the oxide shell (e.g., a cerium-zirconium oxide shell) size, thickness and composition can be tuned based on the synthesis conditions; and (3) the composition and size of the encapsulated NP catalyst can be tuned independent of shell size, composition or thickness. The NP catalyst can be tuned in diameter from about 1-30 nm, and composition of the encapsulated nanoparticle can include, for example, any of the various metal catalyst mentioned above such as Pd, Au, Ag, Pt, and/or alloys thereof. These multicomponent nanomaterial catalysts are therefore highly stable with a high degree of material tunability.

The disclosure provides a method of making a metal oxide shell (either empty or containing a nanoparticle). The method comprises optionally providing a metal core nanoparticle, forming an SiO₂ nanoparticle (optionally encapsulating a metal nanoparticle) by base-catalyzed hydrolysis of tetraethylorhosilicate (TEOS) in ethanol (EtOH) or other appropriate alcohol and water. The SiO₂ particles are washed and dispersed in ethylene glycol and mixed with a metal-oxide precursor in an aqueous solution. The SiO2 particle (optionally containing a metal nanoparticle) serves as a template for generating the nano-shell.

The SiO₂ particles (or other non-metallic particles, e.g., polymethacrylate, polystyrene and the like) provide a template that can have a controlled size. The templates are useful for defining a size and also structure of the oxide shell. Different metal oxides can be easily incorporated onto the templates.

Furthermore, the physical confinement of a metal nanoparticle within a SiO₂ template allows them to remain intact during different physical and chemical processes. The hollow oxide nanosphere are highly desirable for a wide range of applications, particularly in the field of catalysis.

Accordingly, various aspect of the method are tunable to obtain a desired sphere size, encaspulated particle size, pore size and the like to obtain a wide variety of function hollow spheres. For example, the nanoparticles can have different sizes; the nanoparticles can be replaced by various metal or metal oxide particles; the inner diameter can be controlled by the diameter of silica and are tunable by various reaction conditions; the thickness and pore size of the mesoporous layer is also adjustable; changing the composition of mesoporous to other metal oxides (e.g., TiO₂, Al₂O₃, CeO₂, Mb₂O₅, MnO₂) is possible.

Any suitable method and device and combinations thereof can be used for calcination, e.g., heating in a furnace or on a hot plate, irradiation with a light source (UV lamp, IR or heat lamp, laser, etc.), combinations of any of these methods, to name just a few. Also, one or more of these steps may optionally be carried out in a reducing atmosphere (e.g., in an H₂/N₂ atmosphere for metals that are prone to undergo oxidation, especially at elevated temperature, such as e.g., Ni) or in an oxidizing atmosphere.

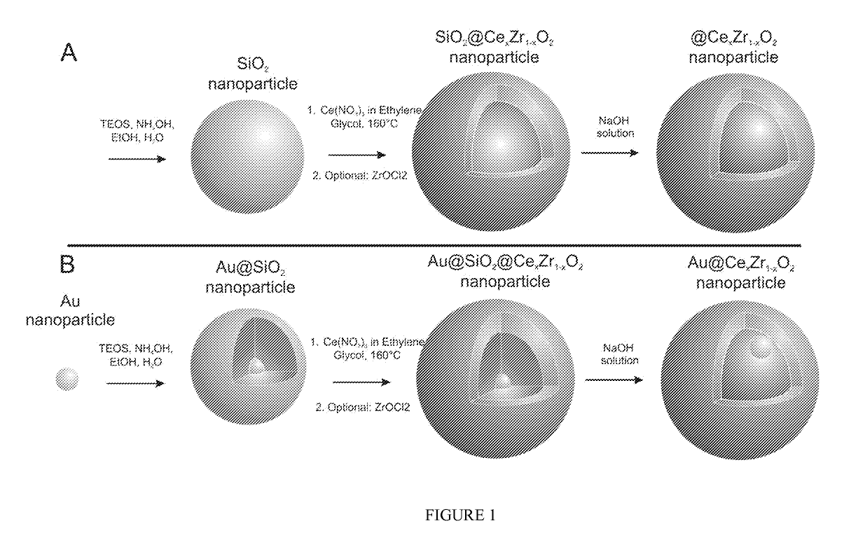

A process for the fabrication of Ce⁻ or Ce/Zr³¹ containing material according to one embodiment is outlined in FIG. 1. As shown in FIG. 1(A), SiO₂ NPs (or “core nanoparticles”) are formed via base-catalyzed hydrolysis of tetraethylorthosilicate (TEOS) in ethanol (EtOH) and water forming SiO₂ NPs of about 50 nm to about 500 nm in size. The SiO₂ NPs are then dispersed in ethylene glycol via sonication and cerium nitrate (aqueous) is added and the solution is stirred for several minutes and then loaded into a Teflon-lined stainless steel autoclave and heated to about 130° C. for about 12 to about 24 hours. An alloy of cerium with zirconium can be generated by adding ZrOCl₂ to the reaction medium. The reaction is sealed and heated to about 180° C. for about 2-12 hours. The obtained SiO2 in a hollow ceria-zirconium oxide shell (SiO₂@CeₓZr₁₋ₓO₂ (1>x>0.5)) are isolated via centrifugation. The product may then be washed several times with ethanol or water and resuspended as appropriate. The SiO₂ cores may be removed via chemical etching with NaOH. In other embodiments, metal NPs can be encapsulated as shown in FIG. 1B by first encapsulating the metal NPs in SiO₂. The remainder of the process is identical to that of the empty ceria-zirconia spheres described above (with reference to FIG. 1A).

In another embodiment, an alkoxide moiety (described above as TEOS) is selected from the group consisting of tetramethoxysilane (TMOS), tetrabutoxysilane (TBOS), tetrapropoxysilane (TPOS), polydiethoxysilane, methyltrimethoxysilane, methyltriethoxysilane, ethyltriethoxysilane, octylpolysilsesquioxane and hexylpolysilsesquioxane. Various surfactants can be used including those selected from the group consisting of polyvinyl alcohol, polyvinyl propanol, Brij 30, Brij 92, Brij 97, sorbitan esters, alkylarylpolyether, alcohol ethoxylates, sodium bis(2-ethylhexyl) sulfosuccinate, and a combination thereof. Furthermore, various metal oxide precursors can be used in the methods including those selected from the group consisting of aluminum bis-ethylacetoacetate monoacetylacetonate, aluminum diacetylacetonate ethyl acetoacetate, aluminum monoacetylacetonate bis-propyl acetoacetate, aluminum monoacetylacetonate bisbutyl acetoacetate, aluminum monoacetylacetonate bis-hexyl acetoacetate, aluminum monoethyl acetoacetate bispropyl acetoacetonate, aluminum monoethyl acetoacetate bisbutyl acetoacetonate, aluminum monoethylacetoacetate bis-hexyl acetoacetonate, aluminum monoethylacetoacetate bisnonylacetoacetonate, aluminum dibutoxide monoacetoacetate, aluminum dipropoxide monoacetoacetate, aluminum butoxide monoethylacetoacetate, aluminum-s-butoxide bis(ethyl acetoacetate), aluminum di-s-butoxide ethylacetoacetate, aluminum-9-octadecenyl acetoacetate diisopropoxide, titanium allylacetoacetate triisopropoxide, titanium di-n-butoxide (bis-2,4-pentanedionate), titanium diisopropoxide (bis-2,4-pentanedionate), titanium diisopropoxide bis(tetramethylheptanedionate), titanium diisopropoxide bis(ethyl acetoacetate), titanium methacryloxyethylacetoacetate triisopropoxide, titanium oxide bis(pentanedionate), zirconium allylacetoacetate triisopropoxide, zirconium di-n-butoxide (bis-2,4-pentanedionate), zirconium diisopropoxide (bis-2,4-pentanedionate), zirconium diisopropoxide bis(tetramethylheptanedionate), zirconium diisopropoxide bis(ethylacetoacetate), zirconium methacryl icoxyethylacetoacetate triisopropoxide, zirconium butoxide (acetylacetate) (bisethylacetoacetate), and iron acetylacetonate. Typically where a metallic nanoparticle is use the metal nanoparticle is a capped noble metal such as Au, Ag, Pt, Pd, Cu, Ni, AuCu or any combination thereof. In one embodiment, the capped nanoparticle comprises an alkylthiol cap. In another embodiment, the alkylthiol comprises from about 1 to 30 carbon atoms.

In a specific aspect, the disclosure provides a method of making a stable mesoporous oxide hollow sphere that encases individual noble metal nanoparticles, the method comprising: sonicating an ethanol solution comprising size monodisperse thiol-capped noble metal nanoparticles and mercaptoundecanoic acid, thereby forming mercaptoundecanoic acid conjugated-nanoparticles; precipitating the mercaptoundecanoic acid conjugated-gold nanoparticles by addition of ammonium hydroxide to the ethanol solution; washing the precipitated mercaptoundecanoic acid conjugated-gold nanoparticles with ethanol and then dissolving the mercaptoundecanoic acid conjugated-gold nanoparticles in water; adding ethanol, ammonium hydroxide and tetraethoxysilane to the aqueous solution of the mercaptoundecanoic acid conjugated-gold nanoparticles, and then stirring the mixture to form gold-silica colloidal particles; centrifuging the mixture containing the gold-silica colloidal particles and re-dispersing the pellet to form an ethanol suspension of gold-silica colloidal particles; adding ethylene glycol a metal precursor such as Ce(NO₃)₃ to the ethanol suspension, followed by stirring and heating; isolating the metal oxide shells encapsulating the gold-silica colloidal particles and calcining the colloidal particles; etching the calcined colloidal particles in sodium hydroxide, thereby removing SiO₂ components and forming hollow spheres containing ligand-free gold nanoparticles.

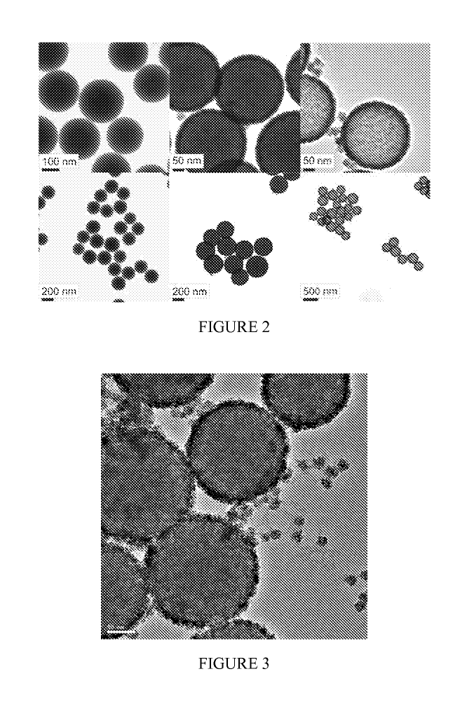

In some cases, reaction of Ce(NO₃)₃ with silica spheres in ethylene glycol can be used to yield a conformal coating of cerium oxide (FIG. 2). Using this method several small, free ceria nanoparticles were present; however, most of the ceria was attached to the SiO₂ templates. The interface between the silica and ceria can be clearly observed due to the differences in morphology and cation atomic number. The CeO₂ coatings were polycrystalline with a crystallite size of about 3-5 nm by TEM and about 4 nm by XRD peak-broadening. The overall thickness of the CeO₂ coating is typically about 10-20 nm. Further reaction with ZrOCl₂ yields a ceria-zirconia alloy (FIG. 3). The ceria coatings were mechanically stable after chemical etching of the SiO₂ template to yield hollow spheres before any calcination or annealing steps (FIG. 2). The ability to etch the silica core confirms the presence of porosity in the cerium oxide coating, for catalytic and drug delivery applications.

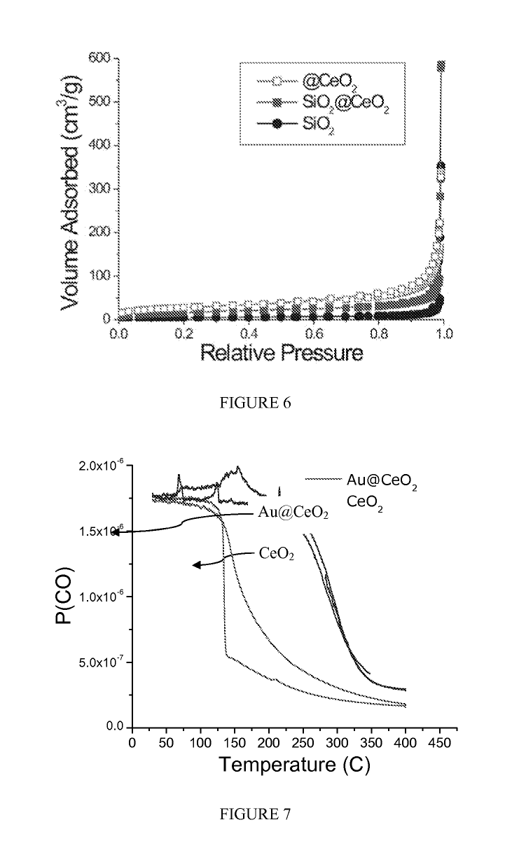

The silica templates had a specific surface area of about 20 m²/g (FIG. 6). The SiO₂@CeO₂ spheres exhibited an increased specific surface area of about 40-50 m²/g and a pore volume of about 0.097 cm³/g. The pore size distribution showed a very broad distribution of pores, suggesting a disordered, nanostructured cerium oxide coating. Upon template removal, the hollow CeO₂ particles had a specific surface area of about 80-100 m²/g and a pore volume of about 0.32 cm³/g. The size of the remaining CeO₂ shell was unchanged following removal of the SiO₂ core. The remarkable increase in pore volume appears to be due to the hollow interiors of the CeO₂ spheres based on the pore size distribution calculated by the well-known Barret-Joyner-Helenda (“BJH”) method.

In addition to SiO₂ NPs, NPs of other substances such as polymethylmethacrylate or polystyrene can be used as a core (or “template”) for formation of nanoparticle shells. In various embodiments, the size of the NPs can range from about 10 to about 500 nanometers.

Core-shell nanoparticles prepared according to the methods described herein can range in size from about 50 nm to about 500 nm.

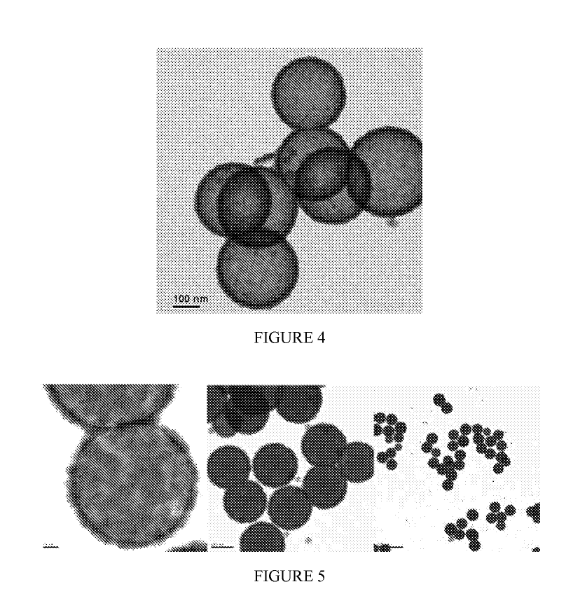

As described herein the compositions of the disclosure provide materials that have utility in various catalytic processes and in drug delivery and therapeutics. For example, high temperature stability is important for many applications including automotive and diesel catalytic conversion. As shown in FIG. 4, cerium oxide spheres are stable up to 750° C. in air. This is close to the upper limit for automotive catalytic converters. The crystallite size as a function of temperature was also examined. A strong increase in crystallite size causes the collapse of the spheres, and occurs around 800° C.

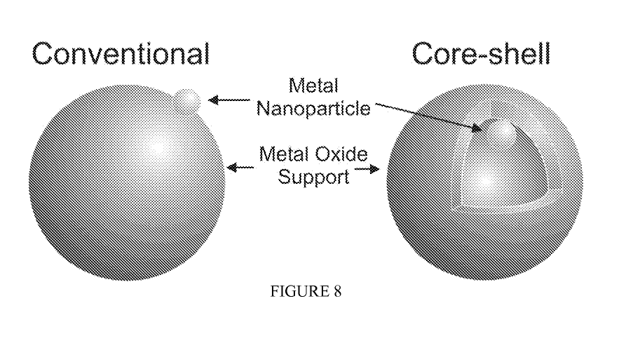

The hollow CeO₂ spheres are active catalysts for CO oxidation (FIG. 7). By incorporating Au NPs in the core of the ceria spheres, the composite particles (Au@CeO₂) are active at lower temperatures than without Au NPs. The ceria spheres without Au NPs oxidize CO fully at approximately 250° C., whereas when Au NPs (about 5 nm diameter) are encapsulated in the ceria spheres, they begin to oxidize CO at approximately 150° C. Cycling several times to about 450° C. would typically cause sintering and a decrease in activity of the Au NP catalysts. However, the catalytic activity of the Au NP catalysts is preserved in the ceria spheres. This indicates that the encapsulation of Au NPs in the ceria spheres indeed prevents temperature-induced sintering.

Thus, the nanomaterials of the disclosure are useful above temperatures in which metal NPs supported by cerium-zirconium oxide (i.e., non-encapsulated NPs) are stable. The nanomaterials of the disclosure are stable to about >750° C. This is significantly higher than the typical temperature at which metal NPs sinter and ripen. Furthermore, the synthesis of the material of the disclosure can be conducted in a closed container in solution and thus can be low cost and scalable. The shell thickness and composition and the core material size and composition can also be independently controlled and tuned for particular uses. Thus a core/shell structure, or a catalyst according to these embodiments, has a high degree of synthetic tunability.

In particular embodiments, CeₓZr₁₋ₓO₂ (1>x>0.5) supported metal NP catalysts can be exposed to significantly higher temperatures compared to conventional (non-core-shell) geometries. In this way, these catalysts can be used (1) at high temperatures, or (2) at lower temperatures in a system that at some point is exposed to high temperatures (e.g., catalytic conversion of diesel or auto exhaust).

The hollow CeₓZr₁₋ₓO₂ and metal containing spheres (e.g., M@CeₓZr₁₋ₓO₂ (1>x>0.5)) can be employed in traditional catalytic converter technology. For example, the particles can simply be dispersed in a washcoat solution and coated onto a clay monolith with other catalysts, which is the method currently used in automotive catalytic converter fabrication.

Further, catalyst particles such as the hollow CeₓZr₁₋ₓO₂ and M@CeₓZr₁₋ₓO₂ particles can be employed in other applications for oxidation of hydrocarbons or CO and reduction of nitrogen oxides. For example the output stream of a waste combustion facility or coal power plant could be treated by these catalysts to aid in the chemistry mentioned above or other reactions such as NOₓ reduction.

Also, since cerium oxide has a very low degree of cytotoxicity, these materials can be used for controlled release of cargo in vivo. Controlled release could be realized by the incorporation of a payload (e.g., DNA, pharmacological agents, drug molecules, therapeutic compounds, radioactive compounds, chemotherapy agents, nucleic acids, proteins, MRI contrast agents, preservatives, flavor compounds, smell compounds, colored dye molecules, fluorescent dye molecules, organometallic compounds, enzyme molecules, pesticides. fungicides and fertilizers, or other organic molecules), which can be used as pharmaceutical compounds, into cerium oxide spheres and subsequent injection into the body. The porosity of the spheres will dictate the rate of release of the encapsulated cargo.

Hollow nanospheres are potentially applicable to drug delivery and imaging. Hollow nanospheres of the disclosure have uniform and stable wall structures with excellent long term stability. Their size can be controlled by using polymer templates for their formation with well-defined diameters accessible from emulsion polymerization. The porosity of the shell is convenient for loading and releasing of drugs or used to contain a heavy element (e.g. metal nanoparticle) or magnetic oxides for X-ray or magnetic contrast reagents. The surface of the hollow silica shell is easily functionalized by grafting biofunctional groups that may combine with targeting proteins, antibodies, cells, or tissues.

A nanostructure of the disclosure can be formulated with a pharmaceutically acceptable carrier, although the nanostructure may be administered alone, as a pharmaceutical composition.

A pharmaceutical composition according to the disclosure can be prepared to include a nanostructure of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers.

Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “effective dose” is meant the quantity of a nanostructure according to the disclosure to sufficiently provide measurable SERS signals. Amounts effective for this use will, of course, depend on the tissue and tissue depth, route of delivery and the like.

Typically, dosages used in vitro may provide useful guidance in the amounts useful for administration of the pharmaceutical composition, and animal models may be used to determine effective dosages for specific in vivo techniques. Various considerations are described, e.g., in Langer, Science, 249: 1527, (1990); Gilman et al. (eds.) (1990), each of which is herein incorporated by reference.

As used herein, “administering an effective amount” is intended to include methods of giving or applying a pharmaceutical composition of the disclosure to a subject that allow the composition to perform its intended function.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 800 of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit.

For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Supplementary active compounds can also be incorporated into the compositions.

The disclosure of International application no. PCT/US09/30687, is incorporated herein for all purposes.

Although the invention has been described in connection with the preferred embodiments, it is to be understood that modifications and variations may be utilized without departing from the principles and scope of the invention, as those skilled in the art will readily understand. Accordingly, such modifications may be practiced within the scope of the following claims.

1. A nanosphere comprising a Group 3 or Group 3/Group 4 metal oxide shell.

2. The nanosphere of claim 1, wherein the shell comprises cerium.

3. The nanosphere of claim 1, wherein the shell comprises cerium and zirconium.

4. The nanosphere of claim 1, wherein the shell comprises CeO₂.

5. The nanosphere of claim 1, wherein the shell comprise ZrO₂.

6. The nanosphere of claim 1, wherein the shell comprise CeₓZr₁₋ₓO₂ wherein 1>x>0.5.

7. The nanosphere of claim 1, further comprising a non-metallic core encapsulated by the shell.

8. The nanosphere of claim 7, wherein the non-metallic core comprises SiO₂, polymethacrylate or polystyrene.

9. The nanosphere of claim 1, further comprising a metallic core encapsulated by the shell.

10. The nanosphere of claim 9, wherein the metal core comprises a metal selected from the group consisting of Au, Pd, Ag, Pt, Ni, Ru, or an alloy thereof.

11. The nanosphere of claim 1, wherein the nanosphere is hollow.

12. The nanosphere of claim 11, wherein the nanosphere comprises a shell of CeO₂ or CeₓZr₁₋ₓO₂ wherein 1>x>0.5.

13. The nanosphere of claim 1, wherein the shell is porous.

14. The nanosphere of claim 9, wherein the nanosphere comprises the general formula M@CeₓZr₁₋ₓO₂, wherein 1>x>0.5, and wherein M comprises a noble metal.

15. A method of making a nanosphere of claim 1 comprising
mixing a core nanoparticle and a reagent comprising a compound of a Group 3 atom under conditions sufficient to form a Group 3 atom-containing shell around the core nanoparticle.

16. The method of claim 15, wherein the Group 3 atom is Ce.

17. The method of claim 15, further comprising mixing the Group 3 atom-containing shell with a compound of a Group 4 atom so as to form a Group 3 atom- and Group 4 atom-containing shell.

18. The method of claim 17, wherein the Group 3 atom is Ce and the Group 4 atom is Zr.

19. The method of claim 15, wherein the core comprises a metal.

20. The method of claim 19, wherein the metal is Au, Pd, Ag, Pt, Ni, Ru, or an alloy thereof.

21. The method of claim 1, wherein the core comprises a metal catalyst.

22. The method of claim 21, wherein the metal catalyst is Au, Pd, Pt, Ru, or RuO₂.

23. The method of claim 15, wherein the core nanoparticle is substantially free of metal.

24. The method of claim 15, further comprising etching away at least part of the core nanoparticle.

25. The method of claim 15, wherein the shell is porous.

26. A catalytic method, comprising carrying out a chemical reaction in the presence of the nanosphere of claim 1, wherein the chemical reaction is catalyzed by the nanosphere or a combination of the nanosphere and a metal core.

27. The method of claim 26, wherein the chemical reaction comprises the oxidation of a hydrocarbon, the oxidation of CO, or the reduction of a nitrogen oxide, or any combination thereof.

28. A catalytic device comprising the nanosphere of claim 1.

29. The catalytic device of claim 28, wherein the device is a catalytic converter.

30. The nanosphere of claim 1, further comprising a pharmaceutical compound encapsulated within the shell.

31. A method of making a nanosphere of claim 2, the method comprising:
forming SiO₂ particles;
suspending the SiO₂ particles in an aqueous mixture of cerium nitrate to coat the SiO₂ particles;
calcining the coated SiO₂ particles to form spheres with a CeO₂ shell; and
etching SiO₂ from the spheres.

32. The method of claim 31, wherein a heterogeneous or homogenous mixture of metal nanoparticles or a metal oxide nanoparticles are included during formation of the SiO₂ particles.

33. The method of claim 32, wherein the metal nanoparticles comprise Au, Ag, Pd, Pt or any combination thereof.

34. The method of claim 31, wherein the nanosphere has an inner diameter of less than 500 nm, the outer layer has a thickness of less than 50 nm, and the outer layer has a pore size of less than 5 nm.
